(19) United States
(12) Patent Application Publication
(10) Pub. No.: US 2011/0224532 A1
(43) Pub. Date: Sep. 15, 2011

(54) PHOTOACOUSTIC BREAST-IMAGE CAPTURING APPARATUS

(75) Inventor: Tsuyoshi TANABE, Ashigarakami-gun (JP)

(73) Assignee: FUJIFILM CORPORATION, Tokyo (JP)

(21) Appl. No.: 13/016,350

(22) Filed: Jan. 28, 2011

(30) Foreign Application Priority Data
Mar. 10, 2010 (JP) 2010-053503

(51) Int. Cl. A61B 5/05

(52) U.S. Cl. 600/407

(57) ABSTRACT

A photoacoustic breast-image capturing apparatus has an image capturing stage on which a breast C1 is placed and a detector provided to the image capturing stage. The detector is constituted of a plurality of capacitive micromachined ultrasonic transducers (cMUT) and light exit surfaces of optical fibers arranged in matrix in X and Y directions with predetermined pitch. The optical fibers are inclined to a side of a patient H by a predetermined angle, and the light exit surfaces of the optical fibers are faced to the patient H. The light beams emitted from the optical fibers on the side of the patient H reach not only the breast C1 but also to the depth of a chest wall C2. A photoacoustic image which is generated from acoustic waves includes information of not only the breast C1 but also the chest wall C2.

### BACKGROUND OF THE INVENTION

1. Field of the Invention

The present invention relates to a photoacoustic breast-image capturing apparatus for obtaining images of breasts using photoacoustic effect.

2. Description of the Related Art

A photoacoustic breast-image capturing apparatus for capturing images of breasts, which are used for breast cancer diagnosis, using photoacoustic effect has recently been attracting an attention. In this apparatus, light (visible light, near-infrared light, or intermediate-infrared light) with a predetermined wavelength is applied to a breast, and acoustic waves generated from the breast which absorbed the optical energy of the applied light are detected, and thereby generating an image (US Patent Application Publication No. 2005/0187471 corresponding to Japanese Patent Laid-Open Publication No. 2005-218684 and US Patent Application Publication No. 2009/0005685 corresponding to Japanese Patent Laid-Open Publication No. 2009-031268).

According to the US Patent Application Publication No. 2005/0187471, a plurality of electroacoustic transducer elements are two-dimensionally arranged at predetermined intervals in the vertical and horizontal directions, and output ends of a plurality of optical fibers are arranged in the gaps between the electroacoustic transducer elements, thereby forming an applicator. The applicator is made into contact with a subject, and acoustic waves generated by light irradiation from the output ends of the optical fibers are detected. This light irradiation is made in almost perpendicular direction to that surface part of the subject which is in contact with the applicator. That is, the light is applied in a direction perpendicular to the flat surface of the two-dimensional array of the electroacoustic transducer elements and the optical fibers. In the US Patent Application Publication No. 2009/0005685, a light guide member which facilitates in-plane radiation of light to a breast and ultrasonic transducers are arranged on the same plane.

In the breast cancer diagnosis, not only a breast but also an area including axillary cavity and chest wall is also captured. When images are captured with the breast held between an image capturing stage and a compression paddle, sometimes the breast needs to be forcibly pulled and squeezed hard with the compression paddle in order to capture the part such as the axillary cavity and the chest wall. This may cause significant discomfort or pain to a patient. However, such problem is not taken into consideration in the US Patent Application Publication Nos. 2005/0187471 and 2009/0005685.

### SUMMARY OF THE INVENTION

An object of the present invention is to provide a photoacostic breast-image capturing apparatus which facilitates accurate breast cancer diagnoses without causing discomfort or pain to patients.

In order to achieve the above and other objects, a photoacoustic breast-image capturing apparatus of the present invention includes an image capturing stage, a first light emission section, a second light emission section, an acoustic wave receiver, and an image processor. A breast of a patient is placed on the image capturing stage. The first light emission section applies light to an area of the breast placed on the image capturing stage. The area of the breast situated on the image capturing stage is referred to as the “placed area”. The second light emission section applies light to a chest wall of the patient. The chest wall situated outside of the placed area of the breast. The acoustic wave receiver receives acoustic waves generated by the application of the light. The image processor forms a photoacoustic image including the breast and the chest wall from an electroacoustic converted signal output from the acoustic wave receiver.

It is preferable that the first and second light emission sections, and the acoustic wave receiver are provided to the image capturing stage.

It is preferable that the photoacoustic breast-image capturing apparatus further includes a compression paddle for compressing the breast on the image capturing stage.

It is preferable that the first and second light emission sections include a plurality of optical fibers attached to the image capturing stage and output the light from a light emission surface of each optical fiber.

It is preferable that the acoustic wave receiver is a plurality of capacitive detection type ultrasonic transducers.

It is preferable that a line of the light emission surfaces of the optical fibers and a line of the capacitive detection type ultrasonic transducers are alternately arranged.

It is preferable that the second light emission section is faced to the patient's side.

It is preferable that the second light emission section is placed closer to the patient as compared to the first light emission section.

It is preferable that an application direction of the light from the first light emission section is almost perpendicular to a plane of the image capturing stage on which the breast is placed, and an application direction of the light from the second light emission section is tilted with respect to the plane.

It is preferable that the second light emission section emits the light from its side surface which is perpendicular to the plane.

It is preferable that the image capturing stage includes aside surface, and the second light emission section is disposed on this side surface.

According to the present invention, the light is applied not only to the breast but also to the chest wall, and therefore facilitating accurate breast cancer diagnoses without causing discomfort nor pain to the patients.

### BRIEF DESCRIPTION OF THE DRAWINGS

For more complete understanding of the present invention, and the advantage thereof, reference is now made to the following descriptions taken in conjunction with the accompanying drawings, in which:

FIG. 1 is a perpendicular view illustrating a photoacoustic breast-image capturing apparatus of the present invention;

FIGS. 2A and 2B are explanatory views illustrating orientations of a breast when its images are captured;

FIG. 3 is a plan view illustrating a detector according to a first embodiment;

FIG. 4A is a sectional view taken along an A-A line of FIG. 3, and FIG. 4B is a sectional view taken along a B-B line of FIG. 3;

FIG. 5 is a block diagram illustrating electrical configuration of the photoacoustic breast-image capturing apparatus;

FIG. 6 is an explanatory view illustrating a process of optical scanning;

FIG. 7 is a lateral view of a detector according to a second embodiment;

FIG. 8 is a sectional view of the detector shown in FIG. 7; and

FIG. 9 is a lateral view of a detector according to a third embodiment.

### DESCRIPTION OF THE PREFERRED EMBODIMENTS

### First Embodiment

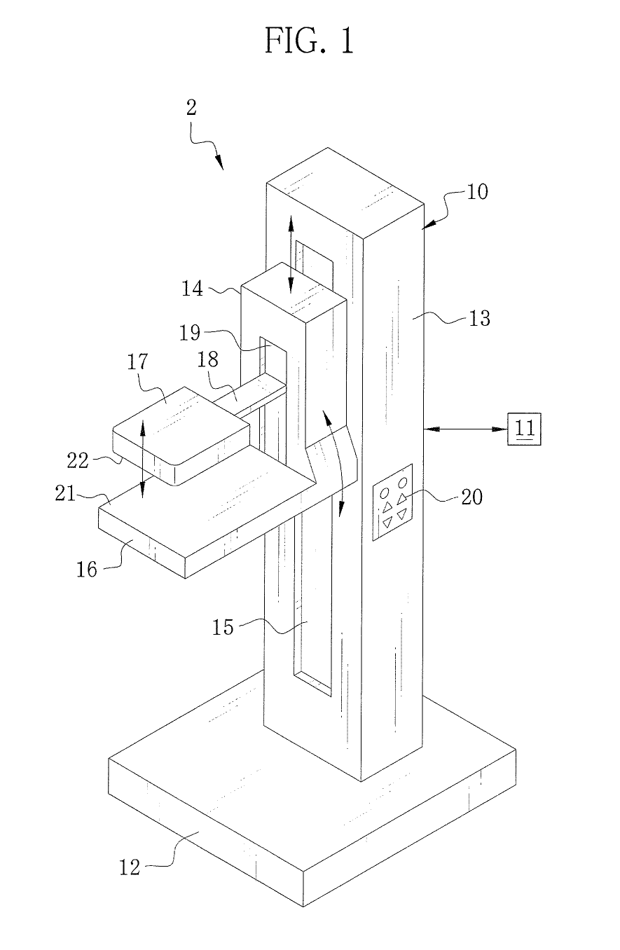

In FIG. 1, a photoacoustic breast-image capturing apparatus 2 includes an image capturing stand 10 and a processor 11. The image capturing stand 10 has a leg portion 12, a fixed support column 13 and a movable support column 14. The leg portion 12 is located on a horizontal floor. The fixed support column 13 is vertically assembled on the leg portion 12. The movable support column 14 is attached to the fixed support column 13. The fixed support column 13 is provided with a guide groove 15 extending along the vertical direction. The movable support column 14 moves up and down in the vertical direction along the guide groove 15 as shown with an arrow. In addition, the movable support column 14 can rotate about with respect to the fixed support column 13 as shown with another arrow.

The movable support column 14 has a rectangular parallelepiped image capturing stage 16 and a compression paddle 17 which are arranged to face with each other. The compression paddle 17 is attached to a guide groove 19 of the movable column 14 via an arm 18, and moves up and down along the guide groove 19 as shown with an arrow. The vertical movement and rotation of the movable support column 14, and the vertical movement of the compression paddle 17 are automatically made by operating operation buttons 20 provided to the fixation support column 13.

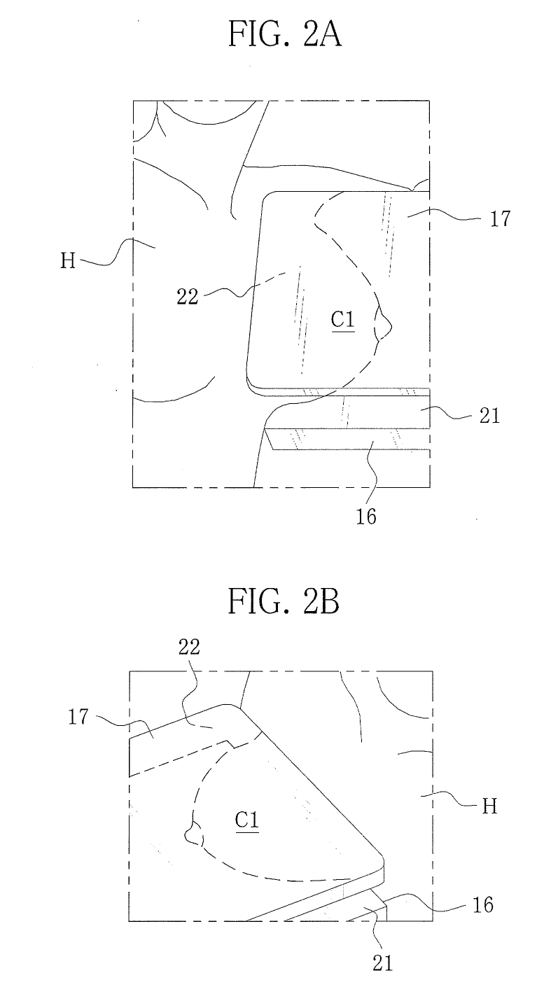

As shown in FIGS. 2A and 2B, a breast C1 of a patient (subject) H is sandwiched between a top surface 21 of the image capturing stage 16 and a bottom surface 22 of the compression paddle 17 facing with each other. FIG. 2A shows the case where a mediolateral oblique (MLO) view is captured. To capture the MLO view, the breast C1 is sandwiched between the image capturing stage 16 and the compression paddle 17 with the top surface 21 tilted about 60 degrees from the horizontal direction, and an image is captured from an oblique or angled direction. FIG. 2B shows the case where a craniocaudal (CC) view is captured. To capture the CC view, the breast C1 is sandwiched between the image capturing stage 16 and the compression paddle 17 with the top surface 21 horizontal, and an image is captured from above.

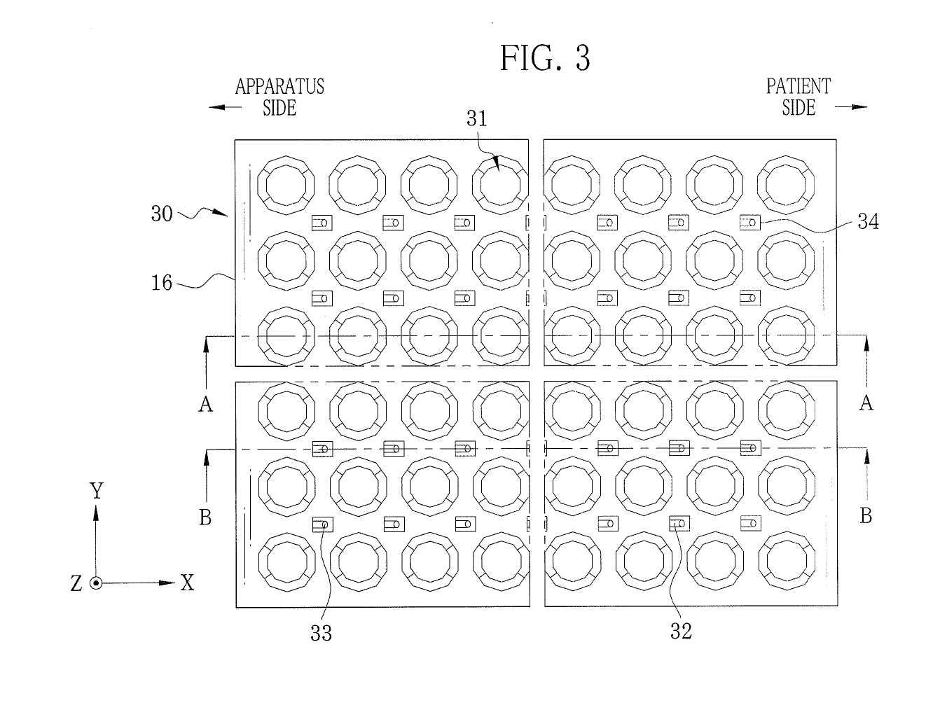

In FIG. 3, a detector 30 is provided on the top surface 21 of the image capturing stage 16. In the detector 30, a plurality of capacitive micromachined ultrasonic transducers (cMUT) 31 and light exit surfaces 33 of optical fibers 32 are arranged in matrix in X and Y directions with predetermined pitch. Note that the X direction is a depth or a longitudinal direction of the image capturing stage 16, the Y direction is a lateral direction of the image capturing stage 16 orthogonal to the X direction, and a Z direction is a thickness direction of the image capturing stage 16.

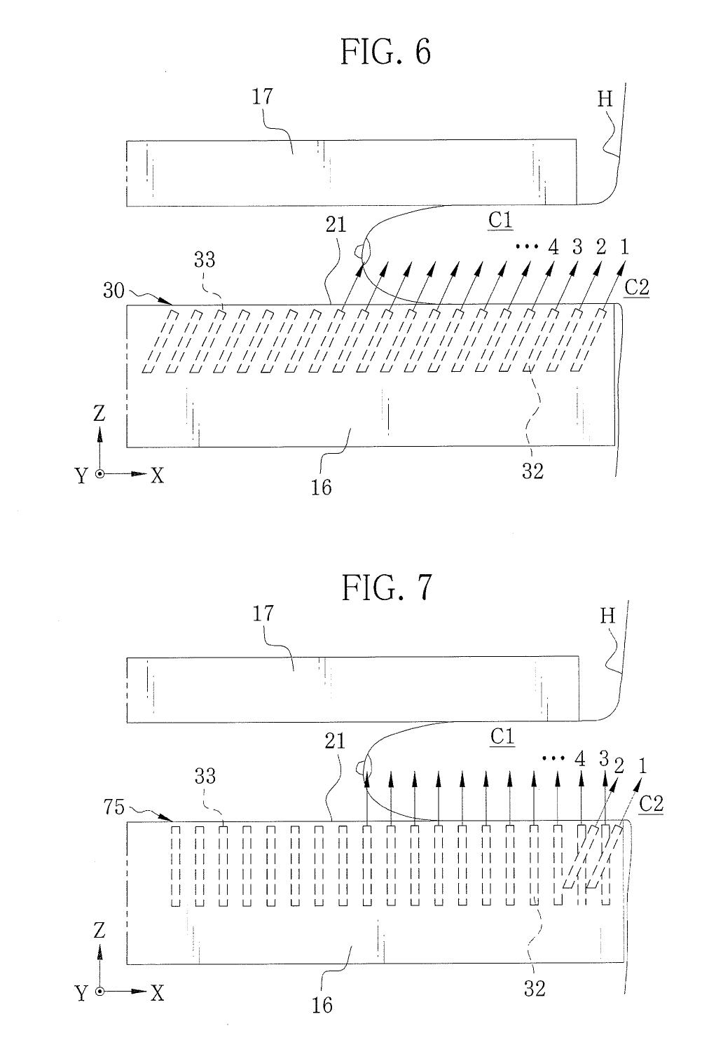

The light exit surface 33 of each optical fiber 32 can be seen through an approximate rectangular opening 34 formed at the center of gap surrounded by four cMUTs 31. Each optical fiber 32 is arranged in the opening 34 with inclined by a predetermined angle to the side of the patient H in the X direction of the image capturing stage 16. Light (visible light, near-infrared light, or intermediate-infrared light) with a predetermined wavelength is obliquely applied from the light exit surface 33 of each optical fiber 32 to the breast C1 and a chest wall C2 (see FIG. 6). The cMUTs 31 receive acoustic waves generated by absorbing energy of the applied light with the breast C1 and the chest wall C2.

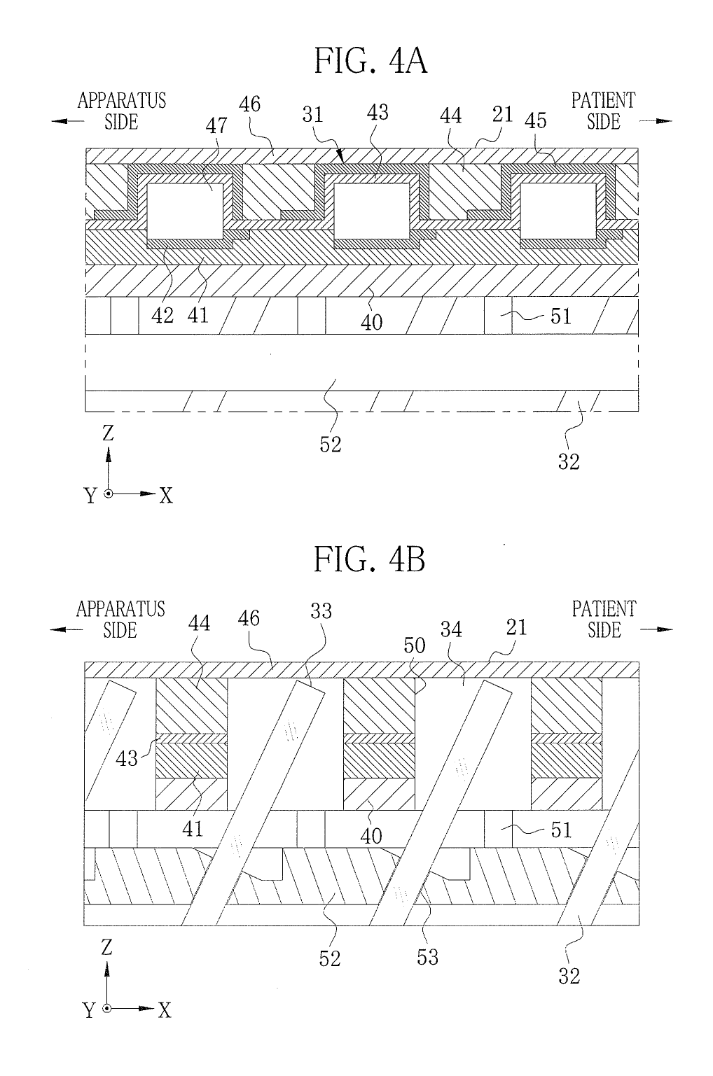

As shown in FIG. 4A, each cMUT 31 is a semiconductor device formed on a silicon substrate 40 by the MEMS technique. On the silicon substrate 40, a first protective layer 41, a lower electrode 42 of the cMUTs 31, a second protective layer 43, a third protective layer 44, an upper electrode 45 of the cMUTs 31, and a fourth protective layer 46 are layered in this order in the Z direction. The fourth protective layer 46 forms the top surface 21, and is made of a transparent material which transmits the light emitted from the light exit surfaces 33 of the optical fibers 32 and the acoustic waves from the breast C1. Note that the fourth protective layer 46 is not shown in FIG. 3.

A cavity layer 47 is formed between the lower electrode 42 and the second protective layer 43. The second protective layer 43 forming the top of the cavity layer 47 functions as a vibrating membrane of the cMUTs 31. The vibrating membrane is arranged parallel to the top surface 21, and vibrates when receiving the acoustic waves. The cMUTs 31 output electroacoustic converted signals according to change in capacitance of each electrode 42, 45 due to vibration of the vibrating membrane, through each electrode 42, 45. It is also possible that the cMUTs 31 transmit ultrasonic waves by vibrating the vibrating membrane when applying voltage to each electrode 42, 45.

In FIG. 4B, the optical fiber 32 is penetrated through a through hole 50 connected to the opening 34. The through holes 50 are formed by, for example, the etching technique used for the semiconductor production process.

A posterior end portion of the optical fiber 32 extending downward from the through hole 50 is held by a retention plate 52 attached to a rear surface of the silicon substrate 40 via a stud 51. The retention plate 52 has retention holes 53 each of which is formed to be inclined by a predetermined angle to the side of the patient H. The posterior end portion of the optical fiber 32 extending downward from the through hole 50 is penetrated through the retention hole 53, and fixed by the retention hole 53 with inclined by the predetermined angle to the side of the patient H while the light exit surface 33 seen from the opening 34.

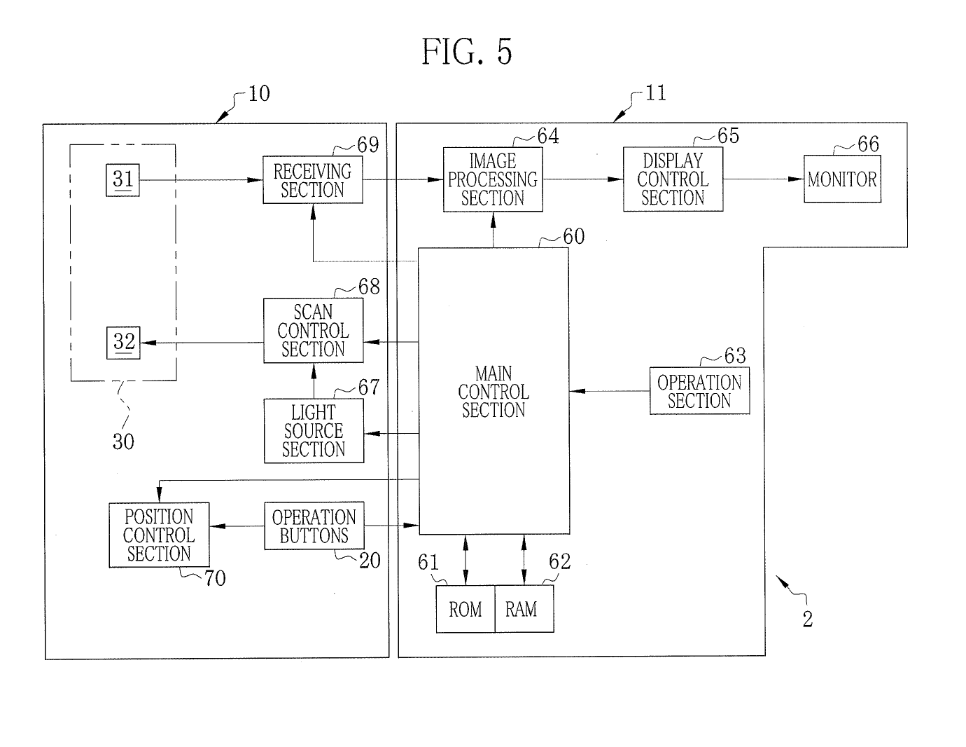

In FIG. 5, the processor 11 has a main control section 60, a ROM 61, a RAM 62, an operation section 63, an image processing section 64, a display control section 65, and a monitor 66. The image capturing stand 10 has a light source section 67, a scan control section 68, a receiving section 69, a position control section 70 in addition to the above-described operation buttons 20 and the detector 30. The position control section 70 controls positions of the movable support column 14 and the compression paddle 17. The main control section 60 takes overall control of operations of the photoacoustic breast-image capturing apparatus 2. The main control section 60 is connected to each component via a data bus, an address bus, or a control line not shown. The ROM 61 stores a variety of programs (OS, application programs and the like) and data (graphic data and the like) for controlling the operation of the processor 11. The main control section 60 reads out necessary programs and data from the ROM 61, and deploys them on the RAM 62 as a work memory to sequentially execute the read out programs. Upon receipt of an operation input signal from the operation section 30 such as a keyboard or from the operation buttons 20 of the image capturing stand 10, the main control section 60 makes each component execute operation corresponding to the input signal.

The light source section 67 is constituted of light emitting elements such as a semiconductor laser, light emitting diode, solid-state laser, gas laser, or the like emitting light with a predetermined wavelength, and introduces the light with the predetermined wavelength into an light entrance surface (not shown) of each optical fiber 32. The scan control section 68 sequentially selects the optical fiber 32 to scan the breast C1 and the chest wall C2 with the light under the control of the main control section 60. The scanning method, that is, the method for projecting the light for scanning is, for example, as follows: each line of the optical fibers 32 horizontal in the Y direction sequentially projects light from the patient H side to the apparatus side in the X direction. In FIG. 6, a number representing the scanning order is given on top of each arrow (light beam).

The receiving section 69 is constituted of an electronic switch and a receiver, and sequentially selects the cMUT 31 adjacent (closest) to the optical fiber 32, which has emitted the light, to receive the acoustic waves from the breast C1 and the chest wall C2 under the control of the main control section 60. The receiving section 69 amplifies the electroacoustic converted signal output from the cMUT 31 which received the acoustic waves from the breast C1 and the chest wall C2, and the amplified signal is subjected to the A/D conversion. The converted signal is further subjected to detection and reception focusing processing, and thereby generating a sound ray signal. The sound ray signal is output to the image processing section 64. In addition to the transmission and reception of the acoustic waves, it is possible that the cMUT 31 transmits the ultrasonic waves and receive the echo waves. In this case, a transmission section for inputting an exciting signal for emitting ultrasonic waves to the cMUT 31 is provided in addition to the receiving section 69.

The image processing section 64 applies a variety of image processing such as interpolation to the sound ray signal from the receiving section 69, and thereby generating a photoacoustic image.

The photoacoustic image is volume data of a plurality of tomographic images captured line by line of the optical fibers 32 parallel to the Y direction. Based on the photoacoustic image generated in the image processing section 64, the display control section 65 displays the above-described tomoographc images along the lines of the optical fibers 32 parallel to the Y direction or other tomographic images in arbitrary directions obtained by volume rendering on the monitor 66. If the cMUT 31 transmits the ultrasonic waves and receives the echo waves, the ultrasonic image obtained by this can also be displayed next to or overlapped with the photoacoustic image on the monitor 66. Note that the tomographic images in arbitrary directions include, for example, the tomographic images taken along the direction parallel to the XZ plane which are same as those obtained by the X-ray mammography. When the tomographic images parallel to the XZ plane are displayed, the images obtained by the X-ray mammography can be compared with ease.

Hereinfafter, the process for diagnosing the breast C1 and the chest wall C2 using the photoacoustic breast-image capturing apparatus 2 having the above-described configuration is explained. First, the breast C1 is positioned between the image capturing stage 16 and the compression paddle 17 and sandwiched therebetween by operating the operation buttons 20, and then it is instructed to start the examination.

Upon receipt of the instruction to start the examination, the main control section 60 drives the light source section 67 to introduce the light to the light entrance surface of each optical fiber 32, and also applies the light to the breast C1 and the chest wall C2 to scan them by controlling the drive of the scan control section 68. In addition, the main control section 60 controls the drive of the receiving section 69 and selectively and sequentially makes the cMUT 31 receive the acoustic waves from the breast C1 and the chest wall C2.

The cMUT 31 outputs the electroacoustic converted signal by receiving the acoustic waves. The electroacoustic converted signal is subjected to the amplification, A/D conversion, detection and reception focusing processing in the receiving section 69, and thereby generating the sound ray signal. The image processing such as interpolation is applied to the sound ray signal in the image processing section 64, and thereby generating the photoacoustic image. The photoacoustic image is then converted into a desired display format in the display control section 65 and displayed as the photoacoustic image on the monitor 66.

In FIG. 6, each optical fiber 32 is arranged to be inclined by the predetermined angle to the side of the patient H, so that the light exit surface 33 faces the side of the patient H. For this configuration, the light beams denoted by the numbers “1” and “2” emitted from the light exit surfaces 33 of the optical fibers 32 on the side of the patient H enter obliquely to the breast C1 and reach to the depth of the chest wall C2. Owing to this, the acoustic waves generated by the application of the light beams denoted by the numbers “1” and “2” contain information not only the breast C1 but also the chest wall C2. For this configuration, the photoacoustic image including the information about the chest wall C2 which is necessary for the breast cancer diagnosis can be easily obtained without forcibly pulling the breast C1 and squeezing it hard with the compression paddle 17.

According to this first embodiment, the optical fibers 32 emitting the light beams denoted by the numbers “1” and “2” constitute the second light emission section and the rest of the optical fibers 32 constitute the first light emission section. Moreover, since all of the optical fibers 32 are inclined by the predetermined angle to the side of the subject H, the structure and the production of the detector 30 can be simplified. Furthermore, since the emission areas of the light beams do not overlap with one another, thermal excitation due to absorption of the light may not accessibly caused, which reduces the burden on the patient H.

Although all of the optical fibers 32 are inclined by the predetermined angle to the side of the patient H in the first embodiment, it is possible that some of the optical fibers 32 are inclined like second and third embodiments shown below. Note that components same as those in the first embodiment are denoted by the same reference numerals and the explanations thereof are omitted.

### Second Embodiment

In FIG. 7, a detector 75 has a configuration that two lines of the optical fibers 32 from the side of the patient H are inclined by a predetermined angle and other optical fibers 32 are parallel to the Z direction so as to be perpendicular to the breast C1. In this case, the scanning starts from the lines of the optical fibers 32 inclined by the predetermined angle, as shown with numbers given on top of arrows (light beams). These two lines of the optical fibers 32 constitute the second light emission section and the rest of the optical fibers 32 constitute the first light emission section.

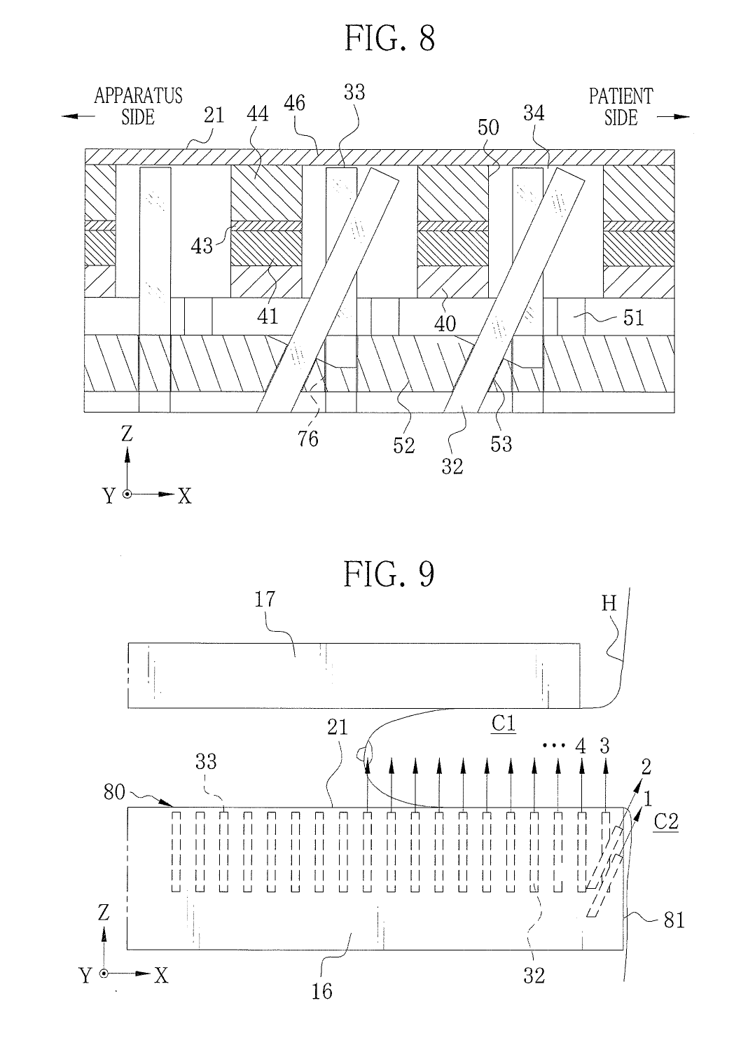

In FIG. 8, the optical fiber 32 inclined by the predetermined angle which performs the scanning first and the optical fiber 32 standing parallel to the Z direction which performs the scanning third are arranged in the same opening 34 and through hole 50 with being shifted from each other in the Y direction. Similarly, the optical fiber 32 inclined by the predetermined angle which performs the scanning second and the optical fiber 32 standing parallel to the Z direction which performs the scanning fourth are arranged in the same opening 34 and through hole 50. The retention plate is formed with a retention hole 76 for the optical fiber 32 standing parallel to the Z direction, in addition to the retention hole 53 for the optical fiber 32 inclined by the predetermined angle. This configuration helps sliming the component as well as simplifying the production process, as compared to the case where the opening 34 and through hole 50 are provided for the optical fiber 32 inclined by the predetermined angle and the optical fiber 32 parallel to the Z direction individually.

When the optical fiber 32 inclined by the predetermined angle and the optical fiber 32 parallel to the Z direction are arranged close to each other in the same opening 34 and through hole 50, the emission areas of the light beams overlap with one another, as shown with the arrows denoted by the numbers “2” and “3” in FIG. 7. In this case, it is preferable that a simple average of the electroacoustic converted signals overlapping with one another is obtained in an area where the light emission areas .overlap and the photoacoustic image is generated. Alternatively, it is also preferable to obtain a weighted average by preferentially weighting the electroacoustic signals including the information about the chest wall C2 and generate the photoacoustic image.

### Third Embodiment

In FIG. 9, a detector 80 has a configuration that some optical fibers 32 inclined by a predetermined angle are arranged on a side surface 81 of the image capturing stage 16 which is diagonal to the patent H, independently from the cMUTs 31 and the optical fibers 32 standing parallel to the Z direction. The side surface 81 of the image capturing stage 16 is formed with openings (not shown) through which the light exit surface 33 of each optical fiber 32 can be seen, and a transparent layer (not shown) same as the fourth protective layer 46. The optical fibers 32 inclined to the side of the patient H constitute the second light emission section.

Since the optical fibers 32 inclined to the side of the patient H by the predetermined angle are provided independently from the cMUTs 31 and the optical fibers 32 standing parallel to the Z direction, the structure and the production of the part where the cMUTs 31 and the optical fibers 32 standing parallel to the Z direction can be simplified. Moreover, since the light emission areas do not overlap, the same effect as the first embodiment can be obtained. Furthermore, since the optical fibers 32 inclined to the side of the patient H by the predetermined angle are provided on the side surface 81 of the image capturing stage 16 which is orthogonal to the patient H, the light exit surfaces 33 can be made closer to the chest wall C2, which enables acquisition of the information about the chest wall C2 efficiently with relatively small optical energy.

It is also possible to combine the first and second embodiments, that is, to incline all of the optical fibers 32 to the side of the patient H, and arrange the optical fibers 32 on the side surface 81 as well.

The lines of the cMUTs and the lines of the optical fibers may separately be produced in different blocks, and each block may alternately be connected to produce the detector. In this case, only the retention plate is used for the blocks of the optical fibers, and the silicon substrate or the first to third protective layers are not necessary.

In the above embodiments, although the cMUTs are used as the acoustic wave receiver, piezoelectric micromachined ultrasonic transducers (pMUT) may also be used. Moreover, a piezo-ceramic thick film such as PZT and the like or a polymer piezoelectric material such as PVDF and the like may also be used. Furthermore, the acoustic wave receiver is not limited to the ultrasonic wave transducer. For example, a magnetostrictor may be used for receiving the acoustic waves.

The arrangement, number and alignment pitch of the cMUTs and the light exit surfaces of the optical fibers, the inclination angle of each optical fiber may appropriately be changed according to the specification of the apparatus. In addition, the order of the light scanning, the selection of the cMUT for receiving the acoustic waves are also appropriately changed. For example, the detector may be provided not only to the image capturing stage but also to the compression paddle so as to perform the light scanning and the reception of the acoustic waves from both sides of the breast. Moreover, it is possible to provide an actuator in which the inclination angle of the optical fibers on the side of the patient H can be changed within a predetermined range according to the shape of the patient H or preference of the operator.

It is also possible that each optical fiber is provided with a light source so that the light scanning is performed by selecting optical fiber one by one upon sequentially turning on and off each light source. When only one light source is used, a movable optical fiber for guiding the light from the light source is provided, and the light scanning is performed by selectively connecting each optical fiber to the movable optical fiber. In addition, the light scanning can be performed using more than one optical fiber instead of using the optical fiber one by one.

Various changes and modifications are possible in the present invention and may be understood to be within the present invention.

What is claimed is:

1. A photoacoustic breast-image capturing apparatus comprising:
an image capturing stage on which a breast of a patient is placed;
a first light emission section for applying light to an area of said breast placed on said image capturing stage (placed area) ;
a second light emission section for applying light to a chest wall of said patient, said chest wall situating outside of said placed area of said breast;
an acoustic wave receiver for receiving acoustic waves generated by the application of said light; and
an image processor for forming a photoacoustic image including said breast and said chest wall from an electroacoustic converted signal output from said acoustic wave receiver.

2. The photoacoustic breast-image capturing apparatus according to claim 1, wherein said first and second light emission sections, and said acoustic wave receiver are provided to said image capturing stage.

3. The photoacoustic breast-image capturing apparatus according to claim 2, further comprising a compression paddle for compressing said breast on said image capturing stage.

4. The photoacoustic breast-image capturing apparatus according to claim 2, wherein said first and second light emission sections include a plurality of optical fibers attached to said image capturing stage and output said light from a light emission surface of each optical fiber.

5. The photoacoustic breast-image capturing apparatus according to claim 4, wherein said acoustic wave receiver is a plurality of capacitive detection type ultrasonic transducers.

6. The photoacoustic breast-image capturing apparatus according to claim 5, wherein a line of said light emission surfaces of said optical fibers and a line of said capacitive detection type ultrasonic transducers are alternately arranged.

7. The photoacoustic breast-image capturing apparatus according to claim 2, wherein said second light emission section is faced to said patient's side.

8. The photoacoustic breast-image capturing apparatus according to claim 7, wherein said second light emission section is placed closer to said patient as compared to said first light emission section.

9. The photoacoustic breast-image capturing apparatus according to claim 2, wherein an application direction of said light from said first light emission section is almost perpendicular to a plane of said image capturing stage on which said breast is placed, and an application direction of said light from said second light emission section is tilted with respect to said plane.

10. The photoacoustic breast-image capturing apparatus according to claim 9, wherein said second light emission section emits said light from its side surface which is perpendicular to said plane.

11. The photoacoustic breast-image capturing apparatus according to claim 9, wherein said image capturing stage includes a side surface, said second light emission section being disposed on said side surface.

12. The photoacoustic breast-image capturing apparatus according to claim 1, wherein said acoustic wave receiver is a plurality of capacitive detection type ultrasonic transducers.
